(19) United States
(12) Patent Application Publication
(10) Pub. No.: US 2024/0382764 A1
(43) Pub. Date: Nov. 21, 2024

(54) OPTIMIZATION OF ATRIAL VENTRICULAR DELAYS WITH CONDUCTION SYSTEM PACING

(71) Applicant: Cardiac Pacemakers, Inc., St. Paul, MN (US)

(72) Inventors: Jessica Burr, Boston, MA (US); Keith L. Hermann, Minneapolis, MN (US); David L. Perschbacher, Blaine, MN (US)

(21) Appl. No.: 18/652,987

(22) Filed: May 2, 2024

Related U.S. Application Data
(60) Provisional application No. 63/466,868, filed on May 16, 2023.

(51) Int. Cl. A61N 1/368; A61N 1/365

(52) U.S. Cl. CPC A61N 1/3682; A61N 1/365; A61N 1/36585

(57) ABSTRACT

A method of operating a cardiac rhythm management (CRM) system includes measuring a baseline PR interval of a cardiac depolarization; measuring one or both of a heart sound and a QRS width for the cardiac depolarization; delivering pacing stimulation according to an atrial sense to ventricular pace interval (AsVp interval) and measuring the one or both of the heart sound and the QRS width for the AsVp interval, wherein the pacing stimulation is delivered using a conduction system pacing (CSP) vector that includes an electrode positioned in an interventricular septum; delivering pacing stimulation according to an atrial pace to ventricular pace interval (ApVp interval) and measuring the one or both of the heart sound and the QRS width for the ApVp interval; and generating a recommended atrial to ventricular delay setting for CSP according to the measured one or both of the heart sounds and the QRS widths.

### CLAIM OF PRIORITY

This application claims the benefit of U.S. Provisional Application No. 63/466,868 filed on May 16, 2023, which is hereby incorporated by reference in its entirety.

### TECHNICAL FIELD

This document relates generally to cardiac rhythm management systems and particularly, but not by way of limitation, to methods, systems, and devices for automatic determination of output settings of a cardiac rhythm management device.

### BACKGROUND

The heart is the center of a person's circulatory system and includes an intrinsic electro-mechanical system for performing two major pumping functions. The left portions of the heart, including a left atrium (LA) and a left ventricle (LV), draw oxygenated blood from the lungs and pump it to body organs to provide the organs with their metabolic need for oxygen. The right portions of the heart, including a right atrium (RA) and a right ventricle (RV), draw deoxygenated blood from the body organs and pump it to lungs where the blood gets oxygenated. These pumping functions result from contractions of the myocardium of the heart. In a normal heart, a sinoatrial (SA) node, the heart's natural pacemaker, generates intrinsic electrical pulses that propagate through an electrical conduction system to various regions of the heart to excite the myocardial tissues of the cardiac muscles. For example, intrinsic electrical pulses originating from the SA node propagate through an atrio-ventricular (AV) node that is between the RA and RV. From the AV node, a specialized intrinsic conduction system is used by the electrical impulses to reach ventricular myocardial tissues, resulting in contraction activities of ventricles. This specialized conduction system includes the His bundle, the right and left conduction bundle branches that extend along the interventricular septum between the RV and LV, and the purkinje fibers that contact the ventricular myocardial tissues.

Coordinated delays of the propagations of the intrinsic electrical pulses in a normal electrical conduction system cause the various portions of the heart to contract in synchrony which results in efficient pumping functions. Heart disease can alter the normal intrinsic conduction paths. A blocked or otherwise abnormal electrical conduction can cause the heart to contract dyssynchronously, resulting in poor hemodynamic performance that may diminish the amount of blood supplied to the heart and the rest of the body.

For example, a block in conduction of the electrical pulses in either of the left bundle branch (LBB) or the right bundle branch (RBB) can cause dyssynchrony among the ventricles (RV and LV) of the heart. Blockage of the normal conduction paths can cause intrinsic electrical pulses to conduct along alternate pathways, which can cause one ventricle to contract later with respect to the other ventricle.

In electrical cardiac pacing therapy, electrical pacing stimulation is generally delivered to the myocardium to improve pumping efficiency of the heart. Pacing energy could be provided to the conduction pathways of the interventricular septum instead of or in addition to the myocardium, but this pacing approach may involve unique timing for sensing and stimulation due to the different conduction paths involved.

### SUMMARY

Device-based stimulation therapy can include techniques to compute an optimum atrial-to-ventricular delay (AV delay) when electrodes are present in the conduction pathways of the interventricular septum of the patient or subject. Example 1 includes subject matter (such as a method of operating a cardiac rhythm management (CRM) system) comprising measuring a baseline P-wave to intrinsic R-wave interval (baseline PR interval) of a cardiac depolarization; measuring one or both of a heart sound and a width of the QRS complex (QRS width) of the cardiac depolarization for the baseline PR interval; delivering pacing stimulation to a ventricle after a sensed atrial event to set an atrial sense to ventricular pace interval (AsVp interval) and measuring the one or both of the heart sound and the QRS width for the AsVp interval, wherein the pacing stimulation is delivered using a conduction system pacing (CSP) vector that includes an electrode positioned in an interventricular septum; delivering pacing stimulation to an atrium to set an atrial pace to ventricular pace interval (ApVp interval) and measuring the one or both of the heart sound and the QRS width for the ApVp interval; and generating a recommended AV delay setting for CSP according to the measured one or both of the heart sounds and the QRS widths.

In Example 2, the subject matter of Example 1 optionally includes changing the AsVp interval for multiple cardiac cycles, and measuring the one or both of the heart sound and the QRS width for the multiple cardiac cycles; changing the ApVp interval for multiple cardiac cycles, and measuring the one or both of the heart sound and the QRS width for the multiple cardiac cycles; and generating the recommended AV delay setting using the one or both of the heart sounds and QRS widths measured for the multiple cardiac cycles.

In Example 3, the subject matter of Example 2 optionally includes measuring both of the heart sound and the QRS width; and generating multiple AV delay settings and presenting the recommended AV delay settings according to magnitude of the heart sound and narrowness of the QRS width.

In Example 4, the subject matter of one or nay combination of Examples 1-3 optionally includes measuring one or both of left ventricular activation times and right ventricular activation times for the baseline PR interval, the AsVp interval, and the ApVp interval; and generating the recommended AV delay according to one or more of the measured heart sounds, the measured QRS widths, and the measured one or both of the left ventricular activation times and the right ventricular activation times.

In Example 5, the subject matter of Example 4 optionally includes measuring one or both of a ventricular activation time interval between delivery of the pacing stimulation to the ventricle and a peak in a sensed far-field QRS complex, wherein the far-field QRS complex is sensed using a sensing vector that includes a can electrode of an ambulatory medical device of the CRM system; and an interval between delivery of the pacing stimulation to the ventricle and a sensed electrogram signal, wherein the electrogram signal is sensed using a sensing vector that includes an electrode used to deliver the pacing stimulation.

In Example 6, the subject matter of one or any combination of Examples 1-5 optionally includes measuring an electrical impedance of one or more heart chambers of the subject for the baseline PR interval, the AsVp interval, and the ApVp interval; and generating the recommended AV delay according to one or more of the measured heart sounds, the measured QRS widths, and the measured electrical impedance.

In Example 7, the subject matter of one or any combination of Examples 1-6 optionally includes measuring an interval between a sensed intrinsic atrial depolarization and a sensed intrinsic ventricular depolarization (AsVs interval; changing to pacing the atrium to set an interval between a paced atrial depolarization and a sensed intrinsic ventricular depolarization (ApVs interval); measuring the one or both of the heart sound and the QRS width for the AsVs interval and ApVs interval; and generating the recommended AV delay setting according to the one or both of the measured heart sounds and the measured QRS widths for the AsVs interval, the ApVs interval, the AsVp interval, and the ApVp interval.

In Example 8, the subject matter of one or any combination of Examples 1-7 optionally includes measuring one or more heart sounds, and measuring a far-field QRS width using a far-field sensing vector that includes a can electrode of an ambulatory medical device of the CRM system; and generating the recommended AV delay according the far-field QRS width and a magnitude of the one or more heart sounds.

In Example 9, the subject matter of one or any combination of Examples 1-8 optionally includes generating a recommended range for the AV delay setting; and changing the AV delay to an AV delay value within the range according to a change in heart rate of the subject.

Example 10 includes subject matter (such as an ambulatory medical device) or can optionally be combined with one or any combination of Examples 1-9 to include such subject matter, comprising a therapy circuit configured to deliver cardiac pacing stimulation energy when connected to electrodes that include at least one CSP electrode positioned in an interventricular septum of a subject; a cardiac signal sensing circuit configured to sense cardiac signals representative of cardiac activity when connected to the electrodes; a heart sound sensing circuit to produce a heart sound signal; and a control circuit operatively coupled to the therapy circuit, the cardiac signal sensing circuit, and the heart sound circuit. The control circuit is configured to measure a baseline PR interval of a sensed cardiac signal; measure one or both of a heart sound and a QRS width of a cardiac depolarization for the baseline PR interval; deliver pacing stimulation to a ventricle using the at least one CSP electrode to set AsVp interval and measure the one or both of the heart sound and the QRS width for the AsVp interval; deliver pacing stimulation to an atrial electrode to set an ApVp interval and measure the one or both of the heart sound and the QRS width for the ApVp interval; and generate a recommended AV delay setting for CSP according to one or both of the measured heart sounds and the measured QRS widths.

In Example 11, the subject matter of Example 10 optionally includes a control circuit configured to change the AsVp interval for multiple cardiac cycles, and measure the one or more of the heart sound and the QRS width for the multiple cardiac cycles; change the ApVp interval for multiple cardiac cycles, and measure the one or more of the heart sound and the QRS width for the multiple cardiac cycles; and generate the recommended AV delay setting using the measured heart sounds and QRS widths for the multiple cardiac cycles.

In Example 12, the subject matter of one or both of Examples 10 and 11 optionally includes a control circuit configured to measure one or both of left ventricular activation times and right ventricular activation times for the baseline PR interval, the AsVp interval, and the ApVp interval; and generate the recommended AV delay setting according to one or more of the measured heart sounds, the measured QRS widths, and the measured one or both of the left ventricular activation times and the right ventricular activation times.

In Example 13, the subject matter of Example 12 optionally includes a housing and a can electrode formed using the housing, a can electrode formed using the housing, and a control circuit configured to measure one or both of a ventricular activation time interval between delivery of the pacing stimulation to the ventricle and a peak in a sensed far-field QRS complex, wherein the far-field QRS complex is sensed using a sensing vector that includes the can electrode, and an interval between delivery of the pacing stimulation to the ventricle and a sensed electrogram signal, wherein the electrogram signal is sensed using a sensing vector that includes an electrode used to deliver the pacing stimulation to the ventricle.

In Example 14, the subject matter of one or any combination of Examples 10-13 optionally includes a cardiac impedance sensing circuit configured to sense an impedance signal representative of electrical impedance of one or more heart chambers; and control circuit configured to measure impedance for each of the baseline PR interval, the AsVp interval, and the ApVp interval using the impedance signal; and generate the recommended AV delay setting based on one or more of the measured heart sounds, the measured QRS widths, and the measured impedance.

In Example 15, the subject matter of one or any combination of Examples 10-14 optionally includes a control circuit configured to measure an interval between a sensed intrinsic atrial depolarization and a sensed intrinsic ventricular depolarization (AsVs interval); deliver pacing stimulation to an atrium and measure an interval between a paced atrial depolarization and a sensed intrinsic ventricular depolarization (ApVs interval); measure the one or both of the heart sound and the QRS width for the AsVs interval and ApVs interval; and generate the recommended AV delay setting according to the one or both of the measured heart sounds and the measured QRS widths for the AsVs interval, ApVs interval, AsVp interval, and ApVp interval.

In Example 16, the subject matter of one or any combination of Examples 10-15 optionally includes a communication circuit coupled to the control circuit and configured to communicate information wirelessly with a separate device; and a control circuit configured to change the AsVp interval for a first plurality of cardiac cycles; change the ApVp interval for a second plurality of cardiac cycles; measure both of magnitude of the heart sound and the QRS width for the first and second plurality of cardiac cycles; generate multiple recommended AV delay settings according to the measured magnitudes of the heart sound and the QRS widths; and communicate the multiple recommended AV delay settings and the measured magnitudes of the heart sound and the QRS widths to the separate device.

In Example 17, the subject matter of one or any combination of Examples 10-16 optionally includes a housing, a can electrode formed using the housing, and a control circuit configured to measure a magnitude of one or more heart sounds using the heart sound signal; measure a far-field QRS width using a far-field sensing vector that includes the can electrode; and generate the recommended AV delay setting according to the far-field QRS width and the measured magnitude of the one or more heart sounds.

In Example 18, the subject matter of one or any combination of Examples 10-17 optionally includes a control circuit configured to generate a recommended range for the AV delay setting; and change the AV delay setting from a first AV delay setting within the recommended range to a second AV delay within the recommended range according to a change in heart rate.

Example 19 includes subject matter (such as a programming device for an ambulatory medical device (AMD)) or can optionally be combined with one or any combination of Examples 1-18 to include such subject matter, comprising a communication circuit configured to communicate information wirelessly with the AMD; a user interface; and a programming control circuit operatively coupled to the communication circuit and user interface. The programming control circuit is configured to enable conduction system pacing (CSP) in the AMD, wherein the enabling the CSP enables delivery of pacing stimulation by the AMD to a CSP pacing vector; initiate an atrial to ventricular delay (AV delay) test in the AMD for the CSP; receive at least one recommended AV delay setting for the CSP from the AMD using the communication circuit; and present the at least one recommended AV delay setting to a user using the user interface.

In Example 20, the subject matter of Example 19 optionally includes a programming control circuit configured to receive multiple AV delay settings for the CSP from the AMD using the communication circuit; receive multiple measurements of heart sound magnitude and QRS width for the multiple AV delay settings; and present the multiple AV delay settings to the user in an order determined by the measurements of one or both of the heart sound magnitude and QRS width.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example Cardiac Rhythm Management (CRM) system.

FIG. 2 illustrates an example of an ambulatory medical device (AMD) and an environment in which it operates.

FIG. 3 is a block diagram of the electronic circuits of an AMD.

FIG. 4 is a block diagram of an external device that communicates information with an AMD.

FIG. 5 is a flow diagram of a method of operating a CRM system.

### DETAILED DESCRIPTION

Ambulatory medical devices (AMDs) can be used to provide cardiac spacing therapy to a patient. AMDs can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

Conventional right ventricular (RV) pacing therapy provides pacing pulses to the RV via electrodes such as to provide relief to a subject suffering from blockage of normal conduction pathways of the right ventricle. Conduction system pacing (CSP) is the direct pacing of the conduction system of the heart, leading to a more physiological activation of the ventricles alternative to traditional right ventricular pacing. CSP therapy provides pacing pulses at multiple positions within the conduction system (e.g., the His Bundle and the Left Bundle Branch) via electrodes located at these positions, and multiple types of capture can result from pacing at these positions.

The number of pacing electrodes available to an AMD to treat cardiac disease, and the increase in the number of programmable features of AMDs to treat cardiac disease can create an extensive parameter search space for the physician or clinician. The present inventors have recognized, among other things, systems and methods for device-based collection and analysis of data can help reduce the parameter search space.

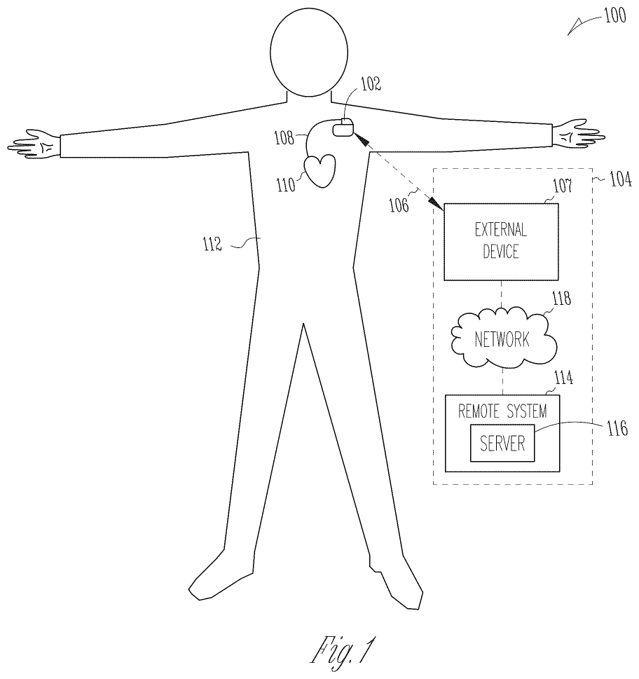

FIG. 1 illustrates portions of an example of a Cardiac Rhythm Management (CRM) system 100 and portions of an environment in which the CRM system 100 can be used. The CRM system 100 can include an AMD 102 that is implantable, an external system 104, and a communication link such as a telemetry link 106. The AMD 102 can include an electronic unit coupled by a cardiac lead 108, or additional leads, to a heart 110 of a subject 112. Examples of the AMD 102 can include, but are not limited to, pacemakers, pacemaker/defibrillators, cardiac resynchronization devices, cardiac remodeling control devices, and cardiac monitors. In an example, the AMD 102 can be configured to monitor health of the heart 110 and determine one or more abnormalities associated with the heart 110. The AMD 102 can take a necessary action, such as stimulating one or more portions of the heart 110 through the lead 108, to treat the one or more abnormalities.

In an example, the external system 104 can include an external device 107 configured to communicate bi-directionally with the AMD 102 such as through the telemetry link 106. For example, the external device 107 can include a programmer to program the AMD 102 to provide one or more therapies to the heart 110. In an example, the external device 107 can program the AMD 102 to detect presence of a conduction block in a left bundle branch (LBB) of the heart 110 and prevent dyssynchronous contraction of the heart 110 by providing a cardiac resynchronization therapy (CRT) to the heart 110.

In an example, the external device 107 can be configured to transmit data to the AMD 102 through the telemetry link 106. Examples of such transmitted data can include programming instructions for the AMD 102 to acquire physiological data, perform at least one self-diagnostic test (such as for a device operational status), or deliver at least one therapy or any other data. In an example, the AMD 102 can be configured to transmit data to the external device 107 through the telemetry link 106. This transmitted data can include real-time physiological data acquired by the AMD 102 or stored in the AMD 102, therapy history data, an operational status of the AMD 102 (e.g., battery status or lead impedance), and the like. The telemetry link 106 can include an inductive telemetry link or a far-field radio-frequency telemetry link.

In an example, the external device 107 can be a part of a CRM system 100 that can include other devices such as a remote system 114 for remotely programming the AMD 102. In an example, the remote system 114 can be configured to include a server 116 that can communicate with the external device 107 through a telecommunication network 118 such as to access the AMD 102 to remotely monitor the health of the heart 110 or adjust parameters associated with the one or more therapies.

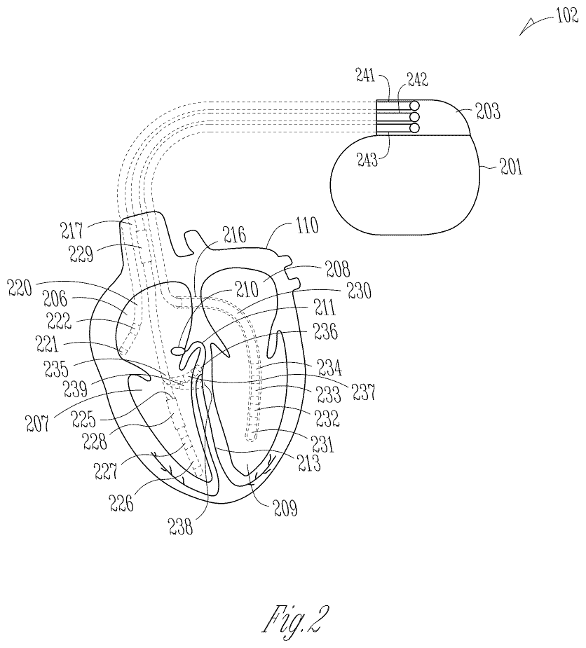

FIG. 2 illustrates an AMD 102 that is implantable and is electrically coupled to a heart 110, such as through one or more leads coupled to the AMD 102 through one or more lead ports, such as first, second, or third lead ports 241, 242, 243 in a header 203 of the AMD 102. In an example, the AMD 102 can include an antenna, such as in the header 203, configured to enable communication with an external system 104 and one or more electronic circuits (e.g., an assessment circuit, etc.) in a hermetically sealed housing (CAN) 201. The AMD 102 illustrates an example ambulatory medical device (or a medical device system) as described herein.

The AMD 102 may include an implantable cardiac monitor (ICM), pacemaker, defibrillator, cardiac resynchronizer, or other subcutaneous AMD or CRM device configured to be implanted in a chest of a subject, having one or more leads to position one or more electrodes or other sensors at various locations in or near the heart 110, such as in one or more of the atria or ventricles. Separate from, or in addition to, the one or more electrodes or other sensors of the leads, the AMD 102 can include one or more electrodes or other sensors (e.g., a pressure sensor, an accelerometer, a gyroscope, a microphone, etc.) powered by a power source in the AMD 102. The one or more electrodes or other sensors of the leads, the AMD 102, or a combination thereof, can be configured detect physiologic information from, or provide one or more therapies or stimulation to, the patient.

The AMD 102 can include one or more electronic circuits configured to sense one or more physiologic signals, such as an electrogram or a signal representing mechanical function of the heart 110. In certain examples, the CAN 201 may function as an electrode such as for sensing or pulse delivery. For example, an electrode from one or more of the leads may be used together with the CAN electrode such as for unipolar sensing of an electrogram or for delivering one or more pacing pulses. A defibrillation electrode (e.g., the first defibrillation coil electrode 228, the second defibrillation coil electrode 229, etc.) may be used together with the electrode of the CAN 201 to deliver one or more cardioversion/defibrillation pulses.

The example lead configuration in FIG. 2 include first, second, and third leads 220, 225, 230 in traditional lead placements in the right atrium (RA) 206, right ventricle (RV) 207, and in a coronary vein 216 (e.g., the coronary sinus) over the left atrium (LA) 208 and left ventricle (LV) 209, respectively. The example in FIG. 2 also shows a fourth lead 235 that can be positioned in the RV 207 near the His bundle 211 in the interventricular septum below the sinoatrial (SA) node 210 or positioned in the RV 207 near the left bundle branches 213. The fourth lead can be a conduction system pacing lead (CSP lead).

Each lead can be configured to position one or more electrodes or other sensors at various locations in or near the heart 110 to detect physiologic information or provide one or more therapies or stimulation. The first lead 220, positioned in the RA 206, can include a first tip electrode 221 located at or near the distal end of the first lead 220 and a first ring electrode 222 located near the first tip electrode 221. The second lead 225 is positioned in the RV 207 and can include a second tip electrode 226 located at or near the distal end of the second lead 225 and a second ring electrode 227 located near the second tip electrode 226. The third lead 230, positioned in the coronary vein 216 over the LV 209, can include a third tip electrode 231 located at or near the distal end of the third lead 230, a third ring electrode 232 located near the third tip electrode 231, and two additional ring electrodes 233, 234.

The fourth lead 235 is positioned in the RV 207. For simplicity of the Figure, the lead 235 is shown optionally positioned near the His bundle 211 or optionally positioned near the left bundle branches 213, but the system may include a separate fourth lead near the His bundle 211 and a fifth lead near the left bundle branches 213. The fourth lead 235 can include a fourth tip electrode 236 located at or near the distal end of the fourth lead 235 for positioning near the His bundle and a fourth ring electrode 237 located near the fourth tip electrode 236. The fourth lead 235 can include a fifth tip electrode 238 located at or near the distal end of the lead for positioning near the left bundle branch and a fifth ring electrode 239 located near the fifth tip electrode 238. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 225 can include a first defibrillation coil electrode 228 located near the distal end of the second lead 225 in the RV 207 and a second defibrillation coil electrode 229 located a distance from the distal end of the second lead 225, such as for placement in or near the superior vena cava (SVC) 217.

Different CRM devices may include different number of leads and lead placements. For example, some CRM devices are single-lead devices having one lead (e.g., RV lead only, RA lead only, CSP lead only, etc.). Other CRM devices are multiple-lead devices having two or more leads (e.g., RA and RV; RA and CSP; RA, RV, and CSP; RV and LV; RA, RV, and LV; etc.). CRM devices adapted for His bundle pacing or left bundle branch pacing may use lead ports designated for LV or RV leads to deliver stimulation to the His bundle 211 or left bundle branches 213.

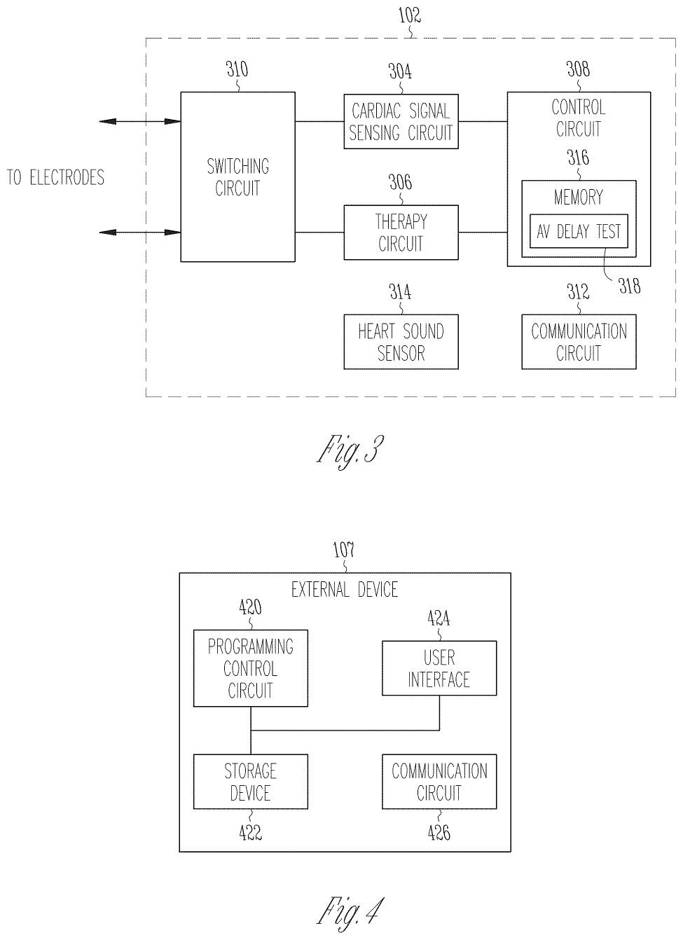

FIG. 3 is a block diagram of portions of electronic circuits of an AMD 102 that is implantable. The AMD 102 can be coupled to multiple implantable electrodes, such as the electrode arrangement described in the example of FIG. 2. The AMD 102 includes a cardiac signal sensing circuit 304, a therapy circuit 306, a heart sound sensor 314, a communication circuit 312, and a control circuit 308. The therapy circuit 306 provides electrical pacing stimulation energy to the heart of the patient when operatively connected to pacing electrodes of the system. The pacing electrodes can include any of the pacing electrodes in FIG. 2, such as electrodes configured for placement in or near the RA, RV, LV, His Bundle, or left bundle branches, and an electrode of the CAN.

The cardiac signal sensing circuit 304 includes one or more sense amplifiers to sense one or both of a voltage signal or a current signal at the electrodes. Cardiac electrical information of the patient can be sensed using the cardiac signal sensing circuit 304. Timing metrics between different features in a sensed electrical signal (e.g., first and second cardiac features, etc.) can be determined, such as by the control circuit 308. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc. Far-field cardiac signals can be sensed using the electrode of the CAN.

Cardiac acceleration information of the patient can be sensed using the heart sound sensor 314. Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively).

The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using heart sound sensor 314 (e.g., an accelerometer or a microphone), providing an output referred to herein as cardiac acceleration information or heart sound information.

The AMD 102 can optionally include a switching circuit 310 to electrically couple different combinations of the electrodes to the therapy circuit 306 and the cardiac signal sensing circuit 304. The switching circuit 310 can configure any combination of the electrodes into a pacing vector to deliver cardiac pacing stimulation energy or configure any combination of the electrodes into a sensing vector to sense a cardiac signal.

The control circuit 308 may include a digital signal processor, application specific integrated circuit (ASIC), field programmable gate array (FPGA), microprocessor, other type of processor, or multiple processors interpreting or executing instructions in software or firmware stored in memory 316 of the control circuit 308. In some examples, the control circuit 308 may include a state machine or sequencer that is implemented in hardware circuits. The control circuit 308 may include any combination of hardware, firmware, or software. The control circuit 308 includes one or more circuits to perform the functions described herein. A circuit may include software, hardware, firmware or any combination thereof. For example, the circuit may include instructions in software executing on the control circuit 308. Multiple functions may be performed by one or more circuits of the control circuit 308. The control circuit 308 uses the communication circuit 312 to communicate information wirelessly with a separate device.

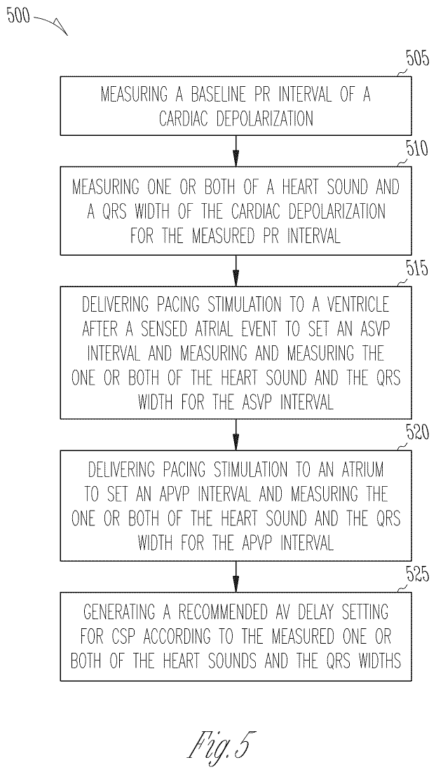

FIG. 4 is a block diagram of portions of an example of an external device 107 (e.g., external device 107 of the neurostimulation system 100 in FIG. 1) to communicate with the AMD 102 of FIG. 3. The external device 107 may be a programming device for the AMD 102. Programming device includes a storage device 422, a programming control circuit 420, a user interface 424, and a communication circuit 426. Programming control circuit 420 may be implemented using an application-specific integrated circuit (ASIC) constructed to perform one or more particular functions, or one or more general-purpose circuits programmed to perform such functions. A general-purpose circuit can include, among other things, a microprocessor or a portion thereof, a microcontroller or portions thereof, and a programmable logic circuit or a portion thereof. The storage device 422 may be a memory integral to the programming control circuit 420, or a separate memory device. Communication circuit 426 communicates information wirelessly with the AMD 102 using near-field inductive wireless signals or far-field radio frequency (RF) signals. The programming device can be used to program pacing therapy parameters and other information in the AMD 102.

FIG. 5 is a flow diagram of a method 500 of operating a CRM system, such as the CRM system 100 in the example of FIG. 1 that includes an AMD 102 (e.g., any of the AMDs in FIGS. 1-3) and an external device 107 (e.g., a programming device) that communicates with the AMD 102. The AMD can be configured (e.g., by programming) for conduction system pacing (CSP) and can be operatively coupled to electrodes configured by shape and size for positioning at a location of the interventricular septum of a patient (e.g., one or more electrodes positioned in the His bundle, the left bundle branch, etc.).

The AMD can sense cardiac electrical signals. A sensed cardiac signal can include a QRS complex. The QRS complex is a waveform produced by depolarization of the ventricles and is composed of a Q-wave, an R-wave, and an S-wave. The Q-wave, R-wave, and S-wave follow a P-wave associated with depolarization of the atria. The interval from the onset of the Q-wave to the termination of the S-wave is sometimes called the QRS width or QRS duration. The time duration of the QRS complex can indicate the efficacy of the cardiac contraction. This can be useful to detect proper beat-to-beat capture of the heart by a device that provides pacing stimulation therapy. A shorter QRS complex would indicate proper capture and a longer QRS complex would indicate a less effective contraction.

The method 500 automatically determines an optimal atrial-to-ventricular (AV) delay for an AMD 102 configured for CSP. The AV delay is the time following a sensed or paced atrial depolarization event at which the AMD 102 is programmed to deliver pacing stimulation to a ventricle if no intrinsic ventricular depolarization is sensed by the AMD 102. If the AV delay is mis-programmed, the chance to capture the left bundle branch or His bundle can be missed. If intrinsic conduction from the atrium arrives late but before the AV delay times out, the resulting ventricular depolarization can have a long QRS complex or a fusion beat can occur, either of which can cause the pumping of the heart to be inefficient. An appropriate AV delay for CSP results in efficient pumping of the heart.

However, delivering pacing stimulation to a conduction path of the interventricular septum complicates determining AV delay because of the difference in the times involved from conventional pacing. For example, there is a delay between pacing the left branch bundle or the HIS bundle and the actual depolarization of the ventricular myocardium. Programming the AV delay can be complicated by the isoelectric interval between the left branch bundle or His bundle and where the myocardial contraction occurs. For example, the interval from stimulation of the His bundle to the depolarization of the ventricular myocardium can be referred to as the His to ventricular (HV) interval. To achieve the same programmed AV delay for conventional RV pacing using an electrode near the RV apex, the AV delay for pacing using an electrode at or near the His bundle would be programmed to a value less than the conventional AV delay by the HV interval to pace the His bundle earlier than pacing the RV apex. For pacing in the left bundle branch (LBB), the AV delay would be programmed to a value less than the conventional AV delay by the LBB to ventricular interval.

The method 500 of FIG. 5 optimizes the atrial-to-ventricular timing of pacing stimulation therapy when CSP electrodes are present. The optimization can be performed using an AV delay test 318 shown included in instructions in memory 316 of the control circuit 308 of the AMD 102 of FIG. 3. Optimization of the atrial-to-ventricular timing can be assessed hemodynamically by the control circuit 308.

At block 505, a baseline P-wave to intrinsic R-wave interval (baseline PR interval) of a cardiac depolarization is measured by the control circuit of the AMD. To determine the baseline PR interval, the AMD senses intrinsic cardiac signals and measures atrial sense to ventricular sense (AsVs) intervals over multiple heart beats. The AMD may be programmed to the NASPE/BPEG defined DDI mode during the measurements. The AV delay can be programmed to an extended AV delay value (e.g., 400 milliseconds, or 400 ms) to promote intrinsic beats to measure the AsVs intervals.

The AMD changes to pacing the atrium and measures atrial pace to ventricular sense (ApVs) intervals over multiple heart beats. The pacing stimulation is delivered using an atrial pacing vector. The atrial pacing vector includes at least one electrode of an atrial lead and can be a bipolar pacing vector (e.g., tip electrode 221 and ring electrode 222 in FIG. 2) or a unipolar pacing vector (e.g., tip electrode 221 and the can electrode in FIG. 2). The AMD may determine the sinus rate from the AsVs intervals and set the pacing rate to faster than the sinus rate (e.g., a pacing rate of 10 beats per minute (10 bpm) above the sinus rate). The AsVs intervals and the ApVs intervals are used to determine the baseline PR interval that can be the native PR interval for the patient. For example, the intervals can be averaged to determine the baseline PR interval.

At block 510, the AMD performs a hemodynamic measurement for the baseline PR interval. For example, the AMD measures one or both of a heart sound parameter and a QRS width as the hemodynamic measurement for the baseline PR interval. The heart sound parameter can be a parameter extracted from a heart sound signal sensed using a heart sound sensor of the AMD (e.g., heart sound sensor 314 in FIG. 3). The heart sound signal can include one or both of an S1 heart sound and an S2 heart sound, and the heart sound parameter can be the magnitude of one or both of the S1 and S2 heart sounds for example.

The QRS width can be measured by the control circuit of the AMD. The control circuit can include signal processing circuitry (e.g., a digital signal processor) to measure the QRS width. In some examples, a far-field sensing vector is used to measure the QRS width. The far-field sensing vector can include a CAN electrode of the AMD and another electrode near the either the right ventricle or left ventricle (e.g., RV tip electrode 226 to CAN electrode in FIG. 2). The heart sound parameter and the QRS width for the baseline PR interval can be determined from heart sound signals and cardiac signals sensed for the AsVs and ApVs intervals. The baseline measurements can be used to assess the native or intrinsic conduction of the patient.

Other measurements can be used to assess the intrinsic conduction of the patient. In some examples, one or both of the left ventricle (LV) activation time and the right ventricle (RV) activation time can be measured for the baseline PR interval. The RV and LV activation times can be determined using the sensed ApVs intervals and can be measured as the time interval from the delivery of the pacing stimulation to the time of the peak of the far-field QRS complex. The interval can be measured using the same far-field sensing vector used to measure the QRS width. The RV and LV activation times can also be measured as the time interval from the delivery of the pacing stimulation to the time of the QRS peak in an electrogram sensed using at least one electrode used to deliver the pacing stimulation.

Another measurement that can be used to assess the intrinsic conduction of the patient is intracardiac impedance. Intracardiac impedance is an electrical impedance measured for one or more heart chambers of the subject. An intracardiac impedance signal can be obtained using electrodes placed within the heart to provide a signal of impedance versus time. For example, in FIG. 2 a cardiac impedance sensor can sense intracardiac impedance of the right ventricle between the electrode 226 placed at the apex of the right ventricle and the defibrillation electrode 229 placed in or near the SVC. A predetermined excitation current is delivered between the electrodes and the impedance is determined from a voltage sensed between the electrodes. The AMD can use one or any combination of the heart sound parameter, the QRS width, the RV activation time, the LN activation time, and the intracardiac impedance to assess the condition of the patient.

At block 515, the AMD enables delivery of pacing stimulation to the ventricle after a sensed atrial event is detected to set an AsVp interval. The pacing stimulation is delivered using a CSP vector that includes an electrode placed in the interventricular septum of the patient. The AMD changes the AV delay for the AsVp intervals over multiple cardiac cycles. When changing the AV delay, the AMD may start by delivering the pacing stimulation using an AV delay slightly shorter than the intrinsic AV delay measured for the AsVs intervals and gradually decrease the AV delay while taking the measurements for the heart sound parameter and QRS width. As an example intended to be non-limiting, the AMD may recurrently decrement the AV delay by 10 ms until the AV delay is in a range from 50 ms-80 ms.

The AV delays for the AsVp intervals are assessed hemodynamically using the same types of measurements as performed for the intrinsic assessment of the baseline PR interval (e.g., one or both of the heart sound parameter and the QRS width). If there is no conduction from the atrium to the ventricles, the control circuit of the AMD may use a predetermined PR interval (e.g., 200 ms) and assess the predetermined PR interval.

At block 520, the AMD enables delivery of pacing stimulation to both the atrium and ventricle to set an atrial pace to ventricular pace interval (ApVp interval). The atrial pacing stimulation may be delivered using the same atrial pacing vector as for the ApVs intervals and the ventricular pacing stimulation may be delivered using the same CSP vector as for the AsVp intervals. The atrial pacing stimulation and the ventricular pacing stimulation are delivered over multiple cardiac cycles. The atrial pacing stimulation may be delivered faster (e.g., 10 bpm faster) than the intrinsic sinus rate measured for the AsVs or the AsVp intervals. The ventricular pacing stimulation may be delivered using the same AV delay intervals as for the AsVp intervals.

The AV delays for the AsVp intervals are assessed hemodynamically using the same types of measurements as performed for the AsVs intervals and the AsVp intervals (e.g., one or more of the heart sound parameter, the QRS width, the RN activation time, the LV activation time, and the intracardiac impedance of the patient). The hemodynamic measurements are recorded for different AV delays for multiple pacing modes. The AMD may store the hemodynamic measurements for the AsVs, AsVp, and ApVp modes.

At block 525, a recommended AV delay setting is generated based on the recorded data. The recommended AV delay setting may be an AV delay that resulted in an increase in magnitude of the S1 or S2 heart sound, narrowing of the QRS width, shortening of an LV or RV activation time, or an improvement in pumping as shows by the intracardiac impedance.

In some examples, the AMD determines the recommended AV delay setting. An external device (e.g., an AMD programmer) uploads the recommended AV delay setting and presents the recommended AV delay to a user (e.g., a clinician or physician). The user can elect to use the recommended AV delay setting using the user interface of the external device. In some examples, the AMD uploads the stored data to the external device (e.g., using the wireless communication circuit) and the external device determines the recommended AV delay setting and presents the recommended AV delay setting to the user.

If multiple types of measurements are made, the types of measurements can be prioritized, and the recommended AV delay setting can be selected according to prioritization of improvements. For example, the heart sound parameter can be prioritized over QRS width. If one AV delay value had an improvement in both heart sound magnitude and QRS width, and a second AV delay value had a better improvement in heart sound magnitude but less of an improvement in QRS width, the second AV delay value would be selected for the recommended AV delay setting based on the prioritization given to heart sound improvement. In another example, if there was no difference in improvement in the highest priority measurement (e.g., heart sound magnitude), the next priority measurement (e.g., QRS width) would be used to determine the recommended AV delay setting. If again there was no difference in the next priority measurement, a third priority measurement could be used. The order of prioritization could be selected by the user.

In another example, a table could be presented with the user interface of the external device. The table may list AV delay settings and the hemodynamic measurements for the AV delay settings. In some examples, the AV delay settings are sorted by the external device according to hemodynamic performance based on the measurements, or a prioritized hemodynamic performance.

In some examples, the recommended AV delay setting is a dynamic AV delay setting that changes with the heart rate of the patient. A sinus rate of the patient can be different at different times of day, and the AV delay setting can change as a function of the sinus rate. In some examples, the recommended AV delay setting is a range of AD delay values, and the AMD sets the AV delay from a first AV delay value within the recommended range to a second AV delay within the recommended range according to a change in heart rate.

The AV delay test and optimization analysis can be run in the clinical setting (e.g., at time of implant or a follow up) and the clinician can set the AV delay according to the results of the AV delay test presented to the clinician. The AV delay analysis can also be performed outside of a clinical setting while the patient is ambulatory. The AV delay analysis can be performed according to a schedule (e.g., a schedule set by the user). In some examples, the AMD recurrently checks a hemodynamic measurement (e.g., QRS width) and runs the AV delay optimization analysis when the hemodynamic measurement is outside a predetermined range.

In some examples, the AMD determines the optimized AV delay setting and resets the AV delay to the new setting while the patient is ambulatory. In some examples, the AMD generates an alert that the current AV delay setting is sub-optimal when the AV delay optimization analysis produces a recommended delay setting that is different than the current AV delay setting of the device or different by more than a predetermined AV delay difference value.

The systems, methods and devices described herein provide device-based collection and analysis of data that help reduce the parameter search space of an AMD. The atrial-to-ventricular timing of electrical pacing therapy is optimized for a particular patient for an AMD that is able to provide the electrical pacing therapy to an interventricular conduction system of the patient.

### Additional Description

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A method of operating a cardiac rhythm management (CRM) system, the method comprising:
measuring a baseline P-wave to intrinsic R-wave interval (baseline PR interval) of a cardiac depolarization of a subject;
measuring one or both of a heart sound and a width of the QRS complex (QRS width) of the cardiac depolarization for the baseline PR interval;
delivering pacing stimulation to a ventricle after a sensed atrial event to set an atrial sense to ventricular pace interval (AsVp interval) and measuring the one or both of the heart sound and the QRS width for the AsVp interval, wherein the pacing stimulation is delivered using a conduction system pacing (CSP) vector that includes an electrode positioned in an interventricular septum;
delivering pacing stimulation to an atrium to set an atrial pace to ventricular pace interval (ApVp interval) and measuring the one or both of the heart sound and the QRS width for the ApVp interval; and
generating a recommended atrial to ventricular delay (AV delay) setting for CSP according to the measured one or both of the heart sounds and the QRS widths.

2. The method of claim 1, including:
changing the AsVp interval for multiple cardiac cycles, and measuring the one or both of the heart sound and the QRS width for the multiple cardiac cycles;
changing the ApVp interval for multiple cardiac cycles, and measuring the one or both of the heart sound and the QRS width for the multiple cardiac cycles; and
wherein the generating the recommended AV delay setting includes generating the recommended AV delay setting using the one or both of the heart sounds and QRS widths measured for the multiple cardiac cycles.

3. The method of claim 2,
wherein the measuring the one or both of the heart sound and the QRS width includes measuring both of the heart sound and the QRS width; and
wherein the generating the recommended AV delay setting includes generating multiple AV delay settings and presenting the recommended AV delay settings according to magnitude of the heart sound and narrowness of the QRS width.

4. The method of claim 1, including:
measuring one or both of left ventricular activation times and right ventricular activation times for the baseline PR interval, the AsVp interval, and the ApVp interval; and
wherein the generating the recommended AV delay setting includes generating the recommended AV delay according to one or more of the measured heart sounds, the measured QRS widths, and the measured one or both of the left ventricular activation times and the right ventricular activation times.

5. The method of claim 4, wherein the measuring the one of both of the left ventricular activation time interval and the right ventricular time interval includes measuring one or both of:
a ventricular activation time interval between delivery of the pacing stimulation to the ventricle and a peak in a sensed far-field QRS complex, wherein the far-field QRS complex is sensed using a sensing vector that includes a can electrode of an ambulatory medical device of the CRM system; and
an interval between delivery of the pacing stimulation to the ventricle and a sensed electrogram signal, wherein the electrogram signal is sensed using a sensing vector that includes an electrode used to deliver the pacing stimulation.

6. The method of claim 1, including:
measuring an electrical impedance of one or more heart chambers of the subject for the baseline PR interval, the AsVp interval, and the ApVp interval; and
wherein the generating the recommended AV delay setting includes generating the recommended AV delay according to one or more of the measured heart sounds, the measured QRS widths, and the measured electrical impedance.

7. The method of claim 1,
wherein the measuring the baseline PR interval includes:
measuring an interval between a sensed intrinsic atrial depolarization and a sensed intrinsic ventricular depolarization (AsVs interval); and
changing to pacing the atrium to set an interval between a paced atrial depolarization and a sensed intrinsic ventricular depolarization (ApVs interval); and
wherein the measuring the one or both of the heart sound and the QRS width of the PR interval includes measuring the one or both of the heart sound and the QRS width for the AsVs interval and ApVs interval; and
wherein the generating the recommended AV delay setting includes generating the recommended AV delay setting according to the one or both of the measured heart sounds and the measured QRS widths for the AsVs interval, the ApVs interval, the AsVp interval, and the ApVp interval.

8. The method of claim 1,
wherein the measuring the one or both of the heart sound and the QRS width includes measuring one or more heart sounds, and measuring a far-field QRS width using a far-field sensing vector that includes a can electrode of an ambulatory medical device of the CRM system; and
wherein the generating the recommended AV delay includes generating the recommended AV delay according the far-field QRS width and a magnitude of the one or more heart sounds.

9. The method of claim 1, wherein generating a recommended AV delay setting includes:
generating a recommended range for the AV delay setting; and
changing the AV delay to an AV delay value within the range according to a change in heart rate of the subject.

10. An ambulatory medical device, the device comprising:
a therapy circuit configured to deliver cardiac pacing stimulation energy when connected to electrodes that include at least one conduction system pacing (CSP) electrode positioned in an interventricular septum of a subject;
a cardiac signal sensing circuit configured to sense cardiac signals representative of cardiac activity when connected to the electrodes;
a heart sound sensing circuit to produce a heart sound signal; and
a control circuit operatively coupled to the therapy circuit, the cardiac signal sensing circuit, and the heart sound circuit; wherein the control circuit is configured to:
measure a baseline P-wave to intrinsic R-wave interval (baseline PR interval) of a sensed cardiac signal;
measure one or both of a heart sound and a width of the QRS complex (QRS width) of a cardiac depolarization for the baseline PR interval;
deliver pacing stimulation to a ventricle using the at least one CSP electrode to set an atrial sense to ventricular pace event interval (AsVp interval) and measure the one or both of the heart sound and the QRS width for the AsVp interval;
deliver pacing stimulation to an atrial electrode to set an atrial pace to ventricular pace interval (ApVp interval) and measure the one or both of the heart sound and the QRS width for the ApVp interval; and
generate a recommended atrial to ventricular delay (AV delay) setting for CSP according to one or both of the measured heart sounds and the measured QRS widths.

11. The device of claim 10, wherein the control circuit is configured to:
change the AsVp interval for multiple cardiac cycles, and measure the one or more of the heart sound and the QRS width for the multiple cardiac cycles;
change the ApVp interval for multiple cardiac cycles, and measure the one or more of the heart sound and the QRS width for the multiple cardiac cycles; and
generate the recommended AV delay setting using the measured heart sounds and QRS widths for the multiple cardiac cycles.

12. The device of claim 10, wherein the control circuit is configured to:
measure one or both of left ventricular activation times and right ventricular activation times for the baseline PR interval, the AsVp interval, and the ApVp interval; and
generate the recommended AV delay setting according to one or more of the measured heart sounds, the measured QRS widths, and the measured one or both of the left ventricular activation times and the right ventricular activation times.

13. The device of claim 12, including:
a housing and a can electrode formed using the housing; and
wherein the control circuit is configured to measure the left ventricular activation times and the right ventricular activation times by measuring one or both of:
a ventricular activation time interval between delivery of the pacing stimulation to the ventricle and a peak in a sensed far-field QRS complex, wherein the far-field QRS complex is sensed using a sensing vector that includes the can electrode; and
an interval between delivery of the pacing stimulation to the ventricle and a sensed electrogram signal, wherein the electrogram signal is sensed using a sensing vector that includes an electrode used to deliver the pacing stimulation to the ventricle.

14. The device of claim 10, including:
a cardiac impedance sensing circuit configured to sense an impedance signal representative of electrical impedance of one or more heart chambers;
wherein the control circuit is configured to:
measure impedance for each of the baseline PR interval, the AsVp interval, and the ApVp interval using the impedance signal; and
generate the recommended AV delay setting based on one or more of the measured heart sounds, the measured QRS widths, and the measured impedance.

15. The device of claim 10, wherein the control circuit is configured to:
measure an interval between a sensed intrinsic atrial depolarization and a sensed intrinsic ventricular depolarization (AsVs interval);
deliver pacing stimulation to an atrium and measure an interval between a paced atrial depolarization and a sensed intrinsic ventricular depolarization (ApVs interval);
measure the one or both of the heart sound and the QRS width for the AsVs interval and ApVs interval; and
generate the recommended AV delay setting according to the one or both of the measured heart sounds and the measured QRS widths for the AsVs interval, ApVs interval, AsVp interval, and ApVp interval.

16. The device of claim 10, including:
a communication circuit coupled to the control circuit and configured to communicate information wirelessly with a separate device; and
wherein the control circuit is configured to:
change the AsVp interval for a first plurality of cardiac cycles;
change the ApVp interval for a second plurality of cardiac cycles;
measure both of magnitude of the heart sound and the QRS width for the first and second plurality of cardiac cycles;
generate multiple recommended AV delay settings according to the measured magnitudes of the heart sound and the QRS widths; and
communicate the multiple recommended AV delay settings and the measured magnitudes of the heart sound and the QRS widths to the separate device.

17. The device of claim 10, including:
a housing and a can electrode formed using the housing; and
wherein the control circuit is configured to:
measure a magnitude of one or more heart sounds using the heart sound signal;
measure a far-field QRS width using a far-field sensing vector that includes the can electrode; and
generate the recommended AV delay setting according to the far-field QRS width and the measured magnitude of the one or more heart sounds.

18. The device of claim 10, wherein the control circuit is configured to:
generate a recommended range for the AV delay setting; and
change the AV delay setting from a first AV delay setting within the recommended range to a second AV delay within the recommended range according to a change in heart rate.

19. A programming device for an ambulatory medical device (AMD), the programming device comprising:
a communication circuit configured to communicate information wirelessly with the AMD;
a user interface; and
a programming control circuit operatively coupled to the communication circuit and user interface; the programming control circuit configured to:
enable conduction system pacing (CSP) in the AMD, wherein the enabling the CSP enables delivery of pacing stimulation by the AMD to a CSP pacing vector;
initiate an atrial to ventricular delay (AV delay) test in the AMD for the CSP;
receive at least one recommended AV delay setting for the CSP from the AMD using the communication circuit; and
present the at least one recommended AV delay setting to a user using the user interface.

20. The programming device of claim 19, wherein the programming control circuit is configured to:
receive multiple AV delay settings for the CSP from the AMD using the communication circuit;
receive multiple measurements of heart sound magnitude and QRS width for the multiple AV delay settings; and
present the multiple AV delay settings to the user in an order determined by the measurements of one or both of the heart sound magnitude and QRS width.
